(19) United States
(12) Patent Application Publication
(10) Pub. No.: US 2023/0025976 A1
(43) Pub. Date: Jan. 26, 2023

(54) PRMT5 Inhibitor for Use In A Method of Treating Psoriasis and Other Autoimmune Conditions

(71) Applicant: Pfizer Inc., New York, NY (US)

(72) Inventors: Meng Li, San Francisco, CA (US); Anthony William Tolcher, San Antonio, TX (US)

(73) Assignee: Pfizer Inc., New York, NY (US)

(21) Appl. No.: 17/758,468

(22) PCT Filed: Jan. 4, 2021
(86) PCT No.: PCT/IB2021/050013

§ 371 Date: Jul. 7, 2022

Related U.S. Application Data
(60) Provisional application No. 63/116,120, filed on Nov. 19, 2020.
(60) Provisional application No. 62/957,925, filed on Jan. 7, 2020.

(51) Int. Cl. A61K 31/519; A61P 17/06

(52) U.S. Cl. CPC A61K 31/519; A61P 17/06

(57) ABSTRACT

Use of PRMT5 inhibitors such as and including (1S,2S,3S,5R)-3-((6-(difluoromethyl)-5-fluoro-1,2,3,4-tetrahydroisoquinolin-8-yl) oxy)-5-(4-methyl-7H-pyrrolo[2,3-d]pyrimidin-7-yl)cyclopentane-1,2-diol:

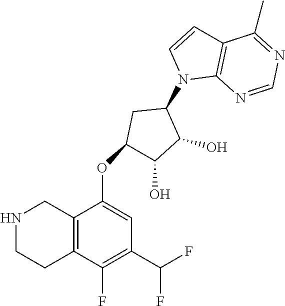 [SMILES: Cc1ncnc2c1ccn2[C@@H]1C[C@H](Oc2cc(C(F)F)c(F)c3c2CNCC3)[C@@H](O)[C@H]1O]

or a pharmaceutically acceptable salt thereof, to treat psoriasis, systemic lupus erythematosus (SLE), rheumatoid arthritis (RA), psoriatic arthritis and other autoimmune conditions or disorders.

### FIELD OF THE INVENTION

The present invention is directed to the use of PRMT5 inhibitors such as and including (1S,2S,3S,5R)-3-((6-(difluoromethyl)-5-fluoro-1,2,3,4-tetrahydroisoquinolin-8-yl)oxy)-5-(4-methyl-7H-pyrrolo [2,3-d]pyrimidin-7-yl)cyclopentane-1,2-diol, having the structure:

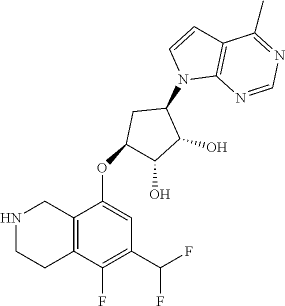 [SMILES: Cc1ncnc2c1ccn2[C@@H]1C[C@H](Oc2cc(C(F)F)c(F)c3c2CNCC3)[C@@H](O)[C@H]1O]

or a pharmaceutically acceptable salt thereof, to treat psoriasis, systemic lupus erythematosus (also called “SLE” or “lupus”), rheumatoid arthritis (RA), psoriatic arthritis and other autoimmune conditions or disorders.

### BACKGROUND OF THE INVENTION

Psoriasis (sometimes referred to as “plaque psoriasis”) is a chronic skin disease affecting approximately about 2-4 percent of the population world-wide. Over seven million people in the United States are affected. While the pathogenesis of psoriasis has not yet been fully elucidated, significant evidence indicates that epidermal changes occur as a secondary response to cellular immune infiltrates in the skin. Psoriasis is characterized by discrete areas of skin inflammation with redness, thickening, intense scaling, and in some cases, itching. The disease has significant impact on the quality of life of affected individuals, both physically and psychologically. Today there is no cure for psoriasis, and treatment is directed at reducing the severity and extent of the psoriatic plaques and the related symptoms. The primary measurement of treatment success used by the U.S. Food and Drug Administration in evaluating products for the treatment of psoriasis is significant overall improvement in psoriasis severity based on Investigators Global Assessment.

Management of psoriasis include topical or systemic medication, phototherapy and various adjunct treatment such as moisturizers and salicylic acid depending on the severity and treatment responses. Systemic medical treatment for more advanced psoriasis include methotrexate, cyclosporine and other small molecule or biologics, such as infliximab, etanercept and ustekinumab. The American academy of dermatology (AAD) has been developing a series of clinical recommendations for the management of psoriasis and psoriatic arthritis (https://www.aad.orq/member/clinical-quality/quidelines). Despite these medical advances, there is still an unmet medical need to develop safer, more effective and convenient medications for psoriasis. Other chronic autoimmune conditions have also been difficult to effectively and/or completely treat SLE, RA, and psoriatic arthritis.

Without being bound by the below-described mechanism of action, PRMT5 and binding partner MEP50 form the methylosome complex that utilizes S adenosylmethionine to transfer methyl groups to arginine, catalyzing both mono- and symmetric di-methylation on substrate residues. PRMT5 methylates multiple protein substrates involved in transcription, cell signalling, mRNA translation, DNA damage, receptor trafficking, protein stability, and pre-mRNA splicing. The most well studied of the PRMT5 substrates are the spliceosomal assembly proteins that regulate pre-mRNA splicing. Mutations in splice sites, splicing factor mutations, and changes in splicing activity have been linked to cancer development and progression. PRMT5 symmetrically di-methylates proteins that regulate pre-mRNA splicing including spliceosomal proteins, SmD1, SmD3 and SmB/B. This methylation increases the affinity of the Sm proteins for the tudor domain of the SMN1 protein, facilitating assembly of small nuclear ribonucleoprotein (snRNP) complexes for proper splice site recognition and recruitment of additional splicing factors. A conditional PRMT5 knockout in mouse neural stem/progenitor cells (NPCs) highlights that PRMT5 function is necessary for proper splice site selection. PRMT5 genetic inhibition leads to increased intron retention and exon skipping in pre-mRNAs resulting in mRNA non-sense mediated decay or alternatively spliced mRNAs. These splicing alterations can reduce expression of proteins or generate alternative “mis-spliced” protein isoforms with unpredictable function in cell cycle regulation, DNA replication and repair, metabolism, and immune pathways.

Alternative splicing and expression of splicing factors have also been associated with autoimmune diseases including: psoriasis (Li J, Yu P. Genome-wide transcriptome analysis identifies alternative splicing regulatory network and key splicing factors in mouse and human psoriasis. Sci Rep. 2018; 8(1):4124. Published 2018 Mar. 7), SLE (Odhams C A, Cortini A, Chen L, et al. Mapping eQTLs with RNA-seq reveals novel susceptibility genes, non-coding RNAs and alternative-splicing events in systemic lupus erythematosus. Hum Mol Genet. 2017; 26(5):1003-1017), and rheumatoid arthritis (Shchetynsky K, Protsyuk D, Ronninger M, Diaz-Gallo L M, Klareskog L, Padyukov L. Gene-gene interaction and RNA splicing profiles of MAP2K4 gene in rheumatoid arthritis. Clin Immunol. 2015; 158(1):19-28). RNA-seq data comparing Tnip knockout mice to human psoriasis patients identified 18 conserved cassette exon events that may be linked to disease (Li et al., supra) in the following genes: ABI1, ARHGAP12, ATP5C1, CTTN, DMN1L, EXOC1, FBLN2, FNBP1, GOLGA2, GOLGA4, MYH11, MYL6, MYO1B, PAM, SEC31A, SLK, SPAG9, and ZMYND11Additionally, twelve splicing factors (CELF1, CELF2, DDX5, MBNL1, MBNL2, NOVA1, PRMT5, PTBP1, RBFOX2, SF3A1, SRRM4, and U2AF1) were identified, including PRMT5, that may contribute to psoriasis by regulating alternatively splicing of genes important in pathways associated with psoriasis. Internal RNA-seq data has identified several of these exons (ABI1, CTTN, EXOC1, GOLGA4, MYL6, PAM, and SEC31A) and splicing factors (MBNL1, PTBP1 and U2AF1) as targets of alternative splicing by PRMT5 inhibitors.

In SLE, 25% of patients have an autoantibody response to Smith antigen (Sm), which includes the spliceosomal proteins SmB, SmD1 and SmD3 (Kalinina O, Louzoun Y, Wang Y, Utset T, Weigert M. Origins and specificity of auto-antibodies in Sm+SLE patients. J Autoimmun. 2018; 90:94-104). PRMT5 symmetrically di-methylates arginines on all three of these proteins, and that methylation increases their antigenicity. Most of the anti-Sm antibodies are directed at these epitopes. Removal of the methylarginine residues from Sm proteins in SLE patients with a PRMT5 inhibitor, may reduce autoantibody response through reduction of the trigger antigen.

PRMT5 also has a reported role in NF-kB signalling, an important pathway involved in chronic inflammation. The p65 subunit of NF-kB transcription factor is directly methylated at several arginine residues (R30, R34 and R174) which impact recruitment of NF-kB to 78% of its target gene promoters, including TRAF1, IL1A, CXCL10 and CXCL11 (Harris D P, Bandyopadhyay S, Maxwell T J, Willard B, DiCorleto P E. Tumor necrosis factor (TNF)-α induction of CXCL10 in endothelial cells requires protein arginine methyltransferase 5 (PRMT5)-mediated nuclear factor (NF)-KB p65 methylation. J Biol Chem. 2014;289(22):15328-15339; Harris D P, Chandrasekharan U M, Bandyopadhyay S, Willard B, DiCorleto P E. PRMT5-Mediated Methylation of NF-κB p65 at Arg174 Is Required for Endothelial CXCL11 Gene Induction in Response to TNF-α and IFN-γ Costimulation. PLoS One. 2016;11(2) published 2016 Feb. 22; and Wei H, Wang B, Miyagi M, et al. PRMT5 dimethylates R30 of the p65 subunit to activate NF-κB. Proc Natl Acad Sci U S A. 2013;110(33):13516-13521). PRMT5 inhibitors could be used to block cytokine induced NF-kB target gene expression, possibly leading to a reduction in inflammation.

U.S. Pat. No. 10,220,037 discloses various compounds and compositions known to inhibit PRMT5 and treat cancer indications. Among these compounds described in U.S. Patent No. 10,220,037 are (1S,2S,3S,5R)-3-((6-(difluoromethyl)-5-fluoro-1,2,3,4-tetrahydroisoquinolin-8-yl) oxy)-5-(4-methyl-7H-pyrrolo[2,3-d]pyrimidin-7-yl)cyclopentane-1,2-diol or a pharmaceutically acceptable salt thereof. Use of these compounds outside of the field of oncology was not known.

### SUMMARY OF THE INVENTION

Disclosed are methods of treating a subject (or a patient) who has an autoimmune condition or disorder, which methods comprise administering to the subject in need thereof a therapeutically effective amount of a PRMT5 inhibitor. The PRMT5 inhibitor can be any PRMT5 inhibitor, or a combination of one or more PRMT5 inhibitors. For instance, one or more PRMT5 inhibitor selected from those known in the art can be used to treat the autoimmune disorders, including but not limited to, (1S,2S,3S,5R)-3-((6-(difluoromethyl)-5-fluoro-1,2,3,4-tetrahydroisoquinolin-8-yl) oxy)-5-(4-methyl-7H-pyrrolo[2,3-d]pyrimidin-7-yl)cyclopentane-1,2-diol, or a pharmaceutically acceptable salt thereof.

In some embodiments, the autoimmune disorder is selected from the group consisting of psoriasis, atopic dermatitis, alopecia areata, ankylosing spondylitis, asthma, type 1 diabetes, multiple sclerosis, celiac disease, scleroderma, hidradenitis suppurativa, vitiligo, dermatomyositis, systemic lupus erythematosus, rheumatoid arthritis, psoriatic arthritis, and inflammatory bowels disease.

In some embodiments, the subject is administered a therapeutically effective amount of a PRMT5 inhibitor enterally (e.g., via oral or rectal route), parenterally (e.g., via intravenous or intraarticular route), or topically. In some embodiments, the subject is administered a therapeutically effective amount from about 0.5 mg to about 120 mg. In some embodiments, the therapeutically effective amount is about 0.5 mg, 1 mg, 2 mg, 4 mg, 6 mg, 8 mg, 10 mg, 16 mg, 32 mg, 60 mg, or 120 mg. In some embodiments, the therapeutically effective amount is administered once daily (QD) or twice daily (BID). In some embodiments, the therapeutically effective amount is administered for 1 to 28 days, 1-6 weeks, 1-4 month, or 1-6 months.

The invention also relates to a PRMT5 inhibitor, in particular (1S,2S,3S,5R)-3-((6-(difluoromethyl)-5-fluoro-1,2,3,4-tetrahydroisoquinolin-8-yl) oxy)-5-(4-methyl-7H-pyrrolo[2,3-d]pyrimidin-7-yl) cyclopentane-1,2-diol, for use in the treatment of an autoimmune condition or disorder; or for use in the manufacture of a medicament useful in the treatment of an autoimmune condition or disorder.

### DETAILED DESCRIPTION OF THE DRAWINGS

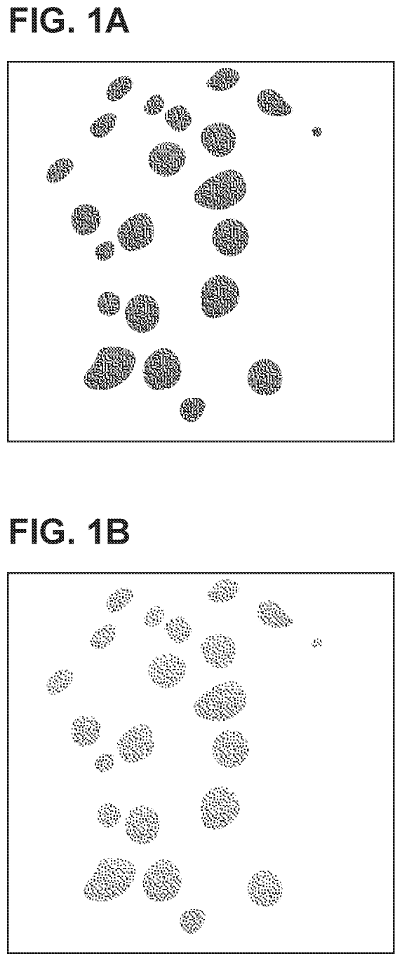

FIG. 1A depicts the skin on a psoriasis patient, front of torso, after receiving treatment of 1 mg twice daily of (1S,2S,3S,5R)-3-((6-(difluoromethyl)-5-fluoro-1,2,3,4-tetrahydroisoquinolin-8-yl) oxy)-5-(4-methyl-7H-pyrrolo[2,3-d]pyrimidin-7-yl)cyclopentane-1,2-diol for two weeks. Psoriasis symptoms are moderate.

FIG. 1B depicts the skin on a psoriasis patient, front of torso, after receiving treatment of 1 mg twice daily of (1S,2S,3S,5R)-3-((6-(difluoromethyl)-5-fluoro-1,2,3,4-tetrahydroisoquinolin-8-yl) oxy)-5-(4-methyl-7H-pyrrolo[2,3-d]pyrimidin-7-yl)cyclopentane-1,2-diol for six weeks. Psoriasis symptoms are mild.

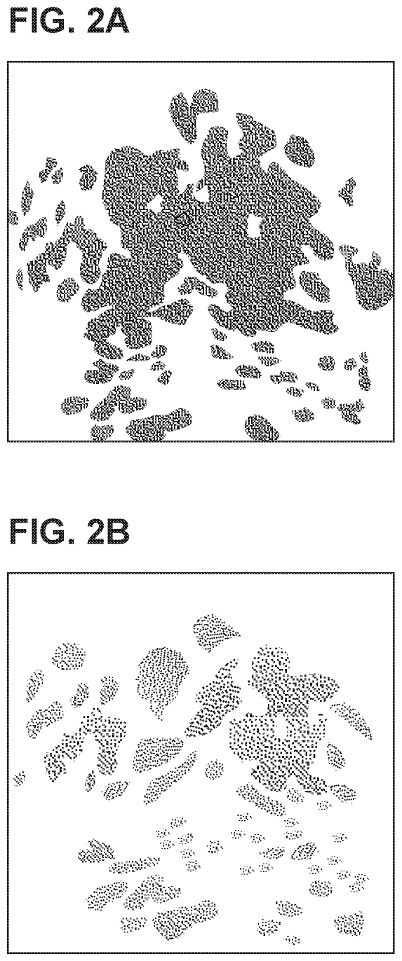

FIG. 2A depicts the skin on a psoriasis patient, back of torso, after receiving treatment of 1 mg twice daily of (1S,2S,3S,5R)-3-((6-(difluoromethyl)-5-fluoro-1,2,3,4-tetrahydroisoquinolin-8-yl) oxy)-5-(4-methyl-7H-pyrrolo[2,3-d]pyrimidin-7-yl)cyclopentane-1,2-diol for two weeks. Psoriasis symptoms are moderate.

FIG. 2B depicts the skin on a psoriasis patient, back of torso, after receiving treatment of 1 mg twice daily of (1S,2S,3S,5R)-3-((6-(difluoromethyl)-5-fluoro-1,2,3,4-tetrahydroisoquinolin-8-yl) oxy)-5-(4-methyl-7H-pyrrolo[2,3-d]pyrimidin-7-yl)cyclopentane-1,2-diol for six weeks. Psoriasis symptoms are mild.

### DETAILED DESCRIPTION OF THE INVENTION

The present invention may be understood more readily by reference to the following detailed description of the preferred embodiments of the invention and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

Compounds useful in connection with the present invention include the PRMT5 inhibitor (1S,2S,3S,5R)-3-((6-(difluoromethyl)-5-fluoro-1,2,3,4-tetrahydroisoquinolin-8-yl)oxy)-5-(4-methyl-7H-pyrrolo [2,3-d]pyrimidin-7-yl)cyclopentane-1,2-diol, and pharmaceutically acceptable salts thereof.

Other PRMT5 inhibitory compounds useful in connection with the present invention include, but not limited to, GSK3326595 (GlaxoSmithKline; CAS No.: 1616392-22-3), JNJ-64619178 (Johnson & Johnson; CAS No.: 2086772-26-9), CTx-034 (Cancer Therapeutics), PRMT5-04 (Auigene Discovery Technologies), EPZ015666 (Epizyme/GlaxoSmithKline), LLY283 and LLY-284 (Eli Lilly; see e.g., Bonday et al., LLY-283, a Potent and Selective Inhibitor of Arginine Methyltransferase 5, PRMT5, with Antitumor Activity. ACS Med Chem Lett. 2018; 9(7): 612-617), PRT543 and PRT811 (Prelude Therapeutics), and others.

According to a first aspect of the invention, there is provided a method of treating an autoimmune condition or disorder in a subject in need thereof comprising administering to the subject a therapeutically effective amount of a PRMT5 inhibitor.

Described below are embodiments (E) of this first aspect of the invention where, for convenience, E1 is identical thereto.

E1. A method of treating an autoimmune disorder in a subject in need thereof comprising administering to the subject a therapeutically effective amount of a PRMT5 inhibitor.

E2. The method according to embodiment E1, wherein the PRMT5 inhibitor is selected from the group consisting of (1S,2S,3S,5R)-3-((6-(difluoromethyl)-5-fluoro-1,2,3,4-tetrahydroisoquinolin-8-yl) oxy)-5-(4-methyl-7H-pyrrolo[2,3-d]pyrimidin-7-yl)cyclopentane-1,2-diol, GSK3326595, JNJ-64619178, CTx-034, PRMT5-04, EPZ015666, LLY283, LLY-284, PRT543, PRT811; and pharmaceutically acceptable salts thereof.

E3. The method according to any one of embodiments E1 to E2, wherein the PRMT5 inhibitor is 1S,2S,3S,5R)-3-((6-(difluoromethyl)-5-fluoro-1,2,3,4-tetrahydroisoquinolin-8-yl)oxy)-5-(4-methyl-7H-pyrrolo [2,3-d]pyrimidin-7-yl)cyclopentane-1,2-diol or a pharmaceutically acceptable salt thereof.

E4. The method according to any one of embodiments E1 to E3, wherein the autoimmune disorder is selected from the group consisting of rheumatoid arthritis, systemic onset rheumatoid arthritis, polyarticular rheumatoid arthritis, enteropathic arthritis, spondyloarthropathy, enteropathic spondylitis, reactive arthritis, axial spondyloarthritis, ankylosing spondylitis, psoriatic arthritis, non-axial spondyloarthritis, osteoarthritis, gouty arthritis, juvenile arthritis, juvenile rheumatoid arthritis, systemic onset juvenile rheumatoid arthritis, periarticular juvenile rheumatoid arthritis, Still's disease, juvenile Reiter's Syndrome, juvenile ankylosing spondylitis, juvenile enteropathic arthritis, juvenile idiopathic arthritis, juvenile psoriatic arthritis, Hashimoto's thyroiditis, autoimmune hemolytic anemia, autoimmune atrophic gastritis of pernicious anemia, autoimmune encephalomyelitis, autoimmune orchitis, vasculitis, Goodpasture's disease, autoimmune thrombocytopenia, sympathetic ophthalmia, myasthenia gravis, Guillain-Barre syndrome, Graves' disease, primary biliary cirrhosis, autoimmune hepatitis, primary sclerosing cholangitis, chronic aggressive hepatitis, non-alcoholic fatty liver disease, non-alcoholic steatohepatitis, cirrhosis, membranous glomerulopathy, focal segmental glomerulosclerosis, Alport syndrome, IgA nephropathy, lupus, systemic lupus, systemic lupus erythematosus (SLE), juvenile SLE, lupus nephritis, Idiopathic pancreatitis, Sjogren's syndrome, myositis polymyositis, dermatomyositis, type I interferonopathies, Aicardi—Goutières syndrome, systemic sclerosis, arteritis, polyarteritis nodosa, multiple sclerosis, relapsing remitting multiple sclerosis, primary progressive multiple sclerosis, secondary progressive multiple sclerosis, bullous pemphigoid, Cogan's syndrome, Wegener's granulomatosis, autoimmune alopecia, vasculitis, nephritis, Bechet's disease, polymyalgia rheumatica, giant cell arteritis, cartilage inflammation, bone degradation, thyroiditis, Type I diabetes, celiac diseases, proctitis, eosinophilic gastroenteritis, eosinophilic esophagitis, mastocytosis, inflammatory bowel disease, eczema, chronic hand eczema, dyshidrotic eczema, chronic itch, atopic dermatitis, contact allergic dermatitis, allergic dermatitis, perioral dermatitis, stasis dermatitis, irritant dermatitis, nummular dermatitis, occupational dermatitis, seborrheic dermatitis, xerotic dermatitis, eyelid dermatitis, diaper dermatitis hand dermatitis, dermatomyositis, neurodermatitis, lichen planus, lichen sclerosis, vitiligo, alopecia areata, pruritis, psoriasis, plaque psoriasis, guttate psoriasis, inverse psoriasis, pustular psoriasis, nail psoriasis, flexural palmoplantar psoriasis, facial psoriasis or erythrodermic psoriasis, rosacea, scleroderma, pemphigus, skin flushing, cutaneous lupus, keloid, sunburn, hypertrophic scar, idiopathic thrombocytopenic thrombotic purpura, ichthyosis, epidermal hyperplasia, acne, epidermolysis bullosa, intertrigo, keratosis pilaris, urticaria, molluscum contagiosum, Netherton syndrome, Vogt-Koyanagi-Harada syndrome, Sweet's syndrome, pityriasis alba, pemphigus, vulvovaginitis, sutton nevus/nevi, post inflammatory hypopigmentation, senile leukoderma, chemical/drug-induced leukoderma, cutaneous lupus erythematosus, discoid lupus, palmoplantar pustulosis, pemphigoid, and hidradenitis suppurativa.

E5. The method according to any one of embodiments E1 to E4, wherein the autoimmune disorder is selected from the group consisting of psoriasis, atopic dermatitis, alopecia areata, ankylosing spondylitis, asthma, type 1 diabetes, multiple sclerosis, celiac disease, scleroderma, hidradenitis suppurativa, vitiligo, dermatomyositis, systemic lupus erythematosus, rheumatoid arthritis, psoriatic arthritis, and inflammatory bowels disease.

E6. The method according to any one of embodiments E1 to E5, wherein the autoimmune disorder is psoriasis.

E7. The method according to any one of embodiments E1 to E5, wherein the autoimmune disorder is atopic dermatitis.

E8. The method according to any one of embodiments E1 to E5, wherein the autoimmune disorder is alopecia areata.

E9. The method according to any one of embodiments E1 to E5, wherein the autoimmune disorder is ankylosing spondylitis.

E10. The method according to any one of embodiments E1 to E5, wherein the autoimmune disorder is asthma.

E11. The method according to any one of embodiments E1 to E5, wherein the autoimmune disorder is type 1 diabetes.

E12. The method according to any one of embodiments E1 to E5, wherein the autoimmune disorder is multiple sclerosis.

E13. The method according to any one of embodiments E1 to E5, wherein the autoimmune disorder is celiac disease.

E14. The method according to any one of embodiments E1 to E5, wherein the autoimmune disorder is scleroderma.

E15. The method according to any one of embodiments E1 to E5, wherein the autoimmune disorder is hidradenitis suppurativa.

E16. The method according to any one of embodiments E1 to E5, wherein the autoimmune disorder is vitiligo.

E17. The method according to any one of embodiments E1 to E5, wherein the autoimmune disorder is dermatomyositis.

E18. The method according to any one of embodiments E1 to E5, wherein the autoimmune disorder is systemic lupus erythematosus.

E19. The method according to any one of embodiments E1 to E5, wherein the autoimmune disorder is rheumatoid arthritis.

E20. The method according to any one of embodiments E1 to E5, wherein the autoimmune disorder is psoriatic arthritis.

E21. The method according to any one of embodiments E1 to E5, wherein the autoimmune disorder is inflammatory bowels disease.

E22. The method according to embodiment E21, wherein the inflammatory bowel disease is

Crohn's disease.

E23. The method according to embodiment E21, wherein the inflammatory bowel disease is ulcerative colitis.

E24. The method according to any one of embodiments E1 to E23, wherein the therapeutically effective amount is from about 0.5 mg to about 120 mg.

E25. The method according to any one of embodiments E1 to E24, wherein the therapeutically effective amount is selected from the group consisting of about: 0.5 mg, 1 mg, 2 mg, 4 mg, 6 mg, 8 mg, 10 mg, 16 mg, 32 mg, 60 mg, 80 mg, and 120 mg.

E26. The method according to any one of embodiments E1 to E25, wherein the therapeutically effective amount is about 1 mg.

E27. The method according to any one of embodiments E1 to E25, wherein the therapeutically effective amount is about 120 mg.

E28. The method according to any one of embodiments E1 to E25, wherein the therapeutically effective amount is about 60 mg.

E29. The method according to any one of embodiments E1 to E25, wherein the therapeutically effective amount is about 32 mg.

E30. The method according to any one of embodiments E1 to E25, wherein the therapeutically effective amount is about 16 mg.

E31. The method according to any one of embodiments E1 to E25, wherein the therapeutically effective amount is about 10 mg.

E32. The method according to any one of embodiments E1 to E25, wherein the therapeutically effective amount is about 8 mg.

E33. The method according to any one of embodiments E1 to E25, wherein the therapeutically effective amount is about 6 mg.

E34. The method according to any one of embodiments E1 to E25, wherein the therapeutically effective amount is about 4 mg.

E35. The method according to any one of embodiments E1 to E25, wherein the therapeutically effective amount is about 2 mg.

E36. The method according to any one of embodiments E1 to E25, wherein the therapeutically effective amount is about 0.5 mg.

E37. The method according to any one of embodiments E1 to E36, wherein the therapeutically effective amount is administered once daily (QD).

E38. The method according to any one of embodiments E1 to E36, wherein the therapeutically effective amount is administered twice daily (QD).

E39. The method according to any one of embodiments E1 to E38, wherein the therapeutically effective amount is administered from 1 to 28 days.

E40. The method according to any one of embodiments E1 to E38, wherein the therapeutically effective amount is administered from 1 to 6 weeks.

E41. The method according to any one of embodiments E1 to E38, wherein the therapeutically effective amount is administered from 1 to 6 months.

E42. The method according to any one of embodiments E1 to E38, wherein the therapeutically effective amount is administered from 1 to 4 months.

E43. The method according to any one of embodiments E1 to E42, wherein the PRMT5 inhibitor is administered enterally, parenterally, or topically.

E44. The method according to E43, wherein said enteral administration is administered orally or rectally.

E45. The method according to E44, wherein said oral administration is administered as a tablet, a capsule, or a liquid dosage form.

E46. The method according to E43, wherein said parenteral administration is administered intravenously or intraarticularly.

E47. The method according to E43, wherein said topical administration is administered as a solution, a cream, an ointment, a gel, a lotion, a suspension, or an emulsion.

E48. The method according to any one of embodiments E1 to E47, further comprising administering an additional therapeutically effective agent.

In some embodiments, when administered orally, (1S,2S,3S,5R)-3-((6-(difluoromethyl)-5-fluoro-1,2,3,4-tetrahydroisoquinolin-8-yl) oxy)-5-(4-methyl-7H-pyrrolo[2,3-d]pyrimidin-7-yl)cyclopentane-1,2-diol or a pharmaceutically acceptable salt thereof can be administered in a tablet, a capsule, or a liquid (suspension, syrup or solution) dosage form.

In some embodiments, when administered parenterally, (1S,2S,3S,5R)-3-((6-(difluoromethyl)-5-fluoro-1,2,3,4-tetrahydroisoquinolin-8-yl) oxy)-5-(4-methyl-7H-pyrrolo[2,3-d]pyrimidin-7-yl) cyclopentane-1,2-diol or a pharmaceutically acceptable salt thereof can be administered intravenously (IV). The IV infusion can be adjusted such that the therapeutically effective amount is delivered through various flow rates.

In some embodiments, when administered parenterally, (1S,2S,3S,5R)-3-((6-(difluoromethyl)-5-fluoro-1,2,3,4-tetrahydroisoquinolin-8-yl) oxy)-5-(4-methyl-7H-pyrrolo[2,3-d]pyrimidin-7-yl) cyclopentane-1,2-diol or a pharmaceutically acceptable salt thereof can be administered as an intraarticular injection.

When applied topically, (1S,2S,3S,5R)-3-((6-(difluoromethyl)-5-fluoro-1,2,3,4-tetrahydroisoquinolin-8-yl) oxy)-5-(4-methyl-7H-pyrrolo[2,3-d]pyrimidin-7-yl)cyclopentane-1,2-diol or a pharmaceutically acceptable salt thereof can be applied in many different pharmaceutical dosage forms all well known to those skilled in the art. These include as a solution, a cream, an ointment, a gel, a lotion, a suspension, or an emulsion, etc.

When administered rectally, (1S,2S,3S,5R)-3-((6-(difluoromethyl)-5-fluoro-1,2,3,4-tetrahydroisoquinolin-8-yl) oxy)-5-(4-methyl-7H-pyrrolo[2,3-d]pyrimidin-7-yl)cyclopentane-1,2-diol or a pharmaceutically acceptable salt thereof can be administered as a suppository which delivers the therapeutically effective amount of the compound as described above.

Regardless of the dosage form, a therapeutically effective amount of (1S,2S,3S,5R)-3-((6-(difluoromethyl)-5-fluoro-1,2,3,4-tetrahydroisoquinolin-8-yl) oxy)-5-(4-methyl-7H-pyrrolo[2,3-d]pyrimidin-7-yl)cyclopentane-1,2-diol is from about 0.5 mg to about 120 mg or more, administered daily, more specifically 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 75 mg, 80 mg, 90 mg, 100 mg, 105 mg, 110 mg, 115 mg, or 120 mg, or other doses, daily.

In some embodiments, a therapeutically effective amount of (1S,2S,3S,5R)-3-((6-(difluoromethyl)-5-fluoro-1,2,3,4-tetrahydroisoquinolin-8-yl) oxy)-5-(4-methyl-7H-pyrrolo[2,3-d]pyrimidin-7-yl) cyclopentane-1,2-diol is at a dose from about 0.05 μg/kg to about 1000 mg/kg, from about 2 mg/kg to about 900 mg/kg, from about 3 mg/kg to about 800 mg/kg, from about 4 mg/kg to about 700 mg/kg, from about 5 mg/kg to about 600 mg/kg, from about 6 mg/kg to about 550 mg/kg, from about 7 mg/kg to about 500 mg/kg, from about 8 mg/kg to about 450 mg/kg, from about 9 mg/kg to about 400 mg/kg, from about 5 mg/kg to about 200 mg/kg, from about 2 mg/kg to about 150 mg/kg, from about 5 mg/kg to about 100 mg/kg, from about 10 mg/kg to about 100 mg/kg, or from about 10 mg/kg to about 60 mg/kg. For example, the therapeutically effective amount is administered to a subject at a dose of at least about 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 3.0 mg/kg, 5.0 mg/kg, 10 mg/kg, 25 mg/kg, 50 mg/kg body weight or more.

In some embodiments, a therapeutically effective amount of (1S,2S,3S,5R)-3-((6-(difluoromethyl)-5-fluoro-1,2,3,4-tetrahydroisoquinolin-8-yl) oxy)-5-(4-methyl-7H-pyrrolo[2,3-d]pyrimidin-7-yl) cyclopentane-1,2-diol is at a dose from about 1 mg/m² to about 3000 mg/m², from about 2 mg/m² to about 2000 mg/m², from about 3 mg/m² to about 1000 mg/m², from about 4 mg/m² to about 750 mg/m², from about 5 mg/m² to about 600 mg/m², from about 6 mg/m² to about 550 mg/m², from about 7 mg/m² to about 500 mg/m², from about 8 mg/m² to about 450 mg/m², from about 9 mg/m² to about mg/m². For example, the therapeutically effective amount is administered to a subject at a dose of at least about 5 mg/m², 10 mg/m², 15 mg/m², 20 mg/m², 25 mg/m², 30 mg/m², 35 mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², 95 mg/m², 100 mg/m², 105 mg/m², 110 mg/m², 115 mg/m², 120 mg/m², 130 mg/m², 135 mg/m², 140 mg/m², 145 mg/m², 150 mg/m², 155 mg/m², 160 mg/m², 165 mg/m², 170 mg/m², 175 mg/m², 180 mg/m², 185 mg/m², 190 mg/m², 195 mg/m², or 200 mg/m².

In some embodiments, administration of the therapeutically effective amount (or dose) of the compounds described herein may take place via a once daily (QD), twice daily (BID), or other dosing regimens. In some embodiments, a daily dose of 16 mg may be administered in one daily dose of 16 mg or in two doses of 8 mg per day. In some embodiments, a daily dose of 6 mg may be administered in one daily dose or 6 mg or in two doses of 3 mg per day. In some embodiments, medication is administered for a period or periods of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. Administration can be administered for longer periods as well and can last for 1, 2, 3, 4, 5, 6, 7, 8, 9, 10, 11, 12, 13, 14, 15, 16, 17, 18, 19, 20, 21, 22, 23, or 24 months, or longer.

While the dosages noted herein are exemplary, the exact dosage and frequency of administration depends on the particular form of (1S,2S,3S,5R)-3-((6-(difluoromethyl)-5-fluoro-1,2,3,4-tetrahydroisoquinolin-8-yl) oxy)-5-(4-methyl-7H-pyrrolo[2,3-d]pyrimidin-7-yl)cyclopentane-1,2-diol or a pharmaceutically acceptable salt thereof used, the severity of the condition being treated, the age, weight, general physical condition of the particular patient, other medication the individual may be taking as is well known to those skilled in the art and can be more accurately determined by measuring the blood level or concentration of (1S,2S,3S,5R)-3-((6-(difluoromethyl)-5-fluoro-1,2,3,4-tetrahydroisoquinolin-8-yl) oxy)-5-(4-methyl-7H-pyrrolo[2,3-d]pyrimidin-7-yl) cyclopentane-1,2-diol or a pharmaceutically acceptable salt thereof in the subject's blood and/or the subject's response to the particular condition being treated.

In certain embodiments of the invention, the amount of (1S,2S,3S,5R)-3-((6-(difluoromethyl)-5-fluoro-1,2,3,4-tetrahydroisoquinolin-8-yl) oxy)-5-(4-methyl-7H-pyrrolo[2,3-d]pyrimidin-7-yl) cyclopentane-1,2-diol or a pharmaceutically acceptable salt thereof used to treat a subject for an autoimmune disorder is less than, or significantly less than (for instance >50% less) the amount of said compound or salt that is needed to treat a cancer. Thus, any side effects and/or toxicities associated with said compound or salt are minimized and mitigated.

As used herein, the singular form “a”, “an”, and “the” include plural references unless indicated otherwise. For example, “a” substituent includes one or more substituents.

The term “about” which used to modify a numerically defined parameter means that the parameter may vary by as much as 10% above or below the stated numerical value for that parameter. For example a dose of about 5 mg/kg should be understood to mean that the dose may vary between 4.5 mg/kg and 5.5 mg/kg, unless otherwise specified.

The term “patient” or “subject” refer to any single subject for which therapy is desired or that is participating in a clinical trial, epidemiological study or used as a control, including humans and mammalian veterinary patients such as cattle, horses, dogs and cats. In certain preferred embodiments, the subject is a human.

The term “treat” or “treating” psoriasis, systemic lupus erythematosus (SLE) or any other autoimmune disorder, condition, or disease as used herein means to administer a therapy according to the present invention to a subject having psoriasis, systemic lupus erythematosus (SLE) or any other autoimmune disorder, condition, or disease to achieve at least one positive therapeutic effect, such as, for example, ameliorating the condition or disease, fewer or less severe psoriatic lesions, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

The term “BID” (or “bid” or “b.i.d.”) means medication is administered twice (two times) a day. For instance, 8 mg BID represents a daily dose of 16 mg administered in a first and second daily 8 mg dose.

The term “QD” (or “qd” or “q.d.”) means medication is administered once (one time) a day

The terms “treatment regimen”, “dosing protocol” and “dosing regimen” are used interchangeably to refer to the dose and timing of administration of each therapeutic agent in a combination of the invention.

“Ameliorating” means a lessening or improvement of one or more symptoms upon treatment with a combination described herein, as compared to not administering the combination. “Ameliorating” also includes shortening or reduction in duration of a symptom.

As used herein, an “effective dosage”, “effective dose”, “effective amount”, or “therapeutically effective amount” of drug, compound or pharmaceutical composition is an amount sufficient to effect any one or more beneficial or desired, including biochemical, histological and/or behavioural symptoms, of the disease, its complications and intermediate pathological phenotypes presenting during development of the disease. For therapeutic use, a “therapeutically effective amount” refers to that amount of a compound being administered which will relieve to some extent one or more of the symptoms of the disorder being treated. An effective dosage can be administered in one or more administrations. For the purposes of this invention, an effective dosage of drug, compound, or pharmaceutical composition is an amount sufficient to accomplish prophylactic or therapeutic treatment either directly or indirectly. As is understood in the clinical context, an effective dosage of drug, compound or pharmaceutical composition may or may not be achieved in conjunction with another drug, compound or pharmaceutical composition.

Pharmaceutically acceptable refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability.

When the solubility of a solid in a solvent is used the ratio of the solid to the solvent is weight/volume (wt/v).

When the % active ingredient of a pharmaceutical formulation is set forth, it is the ratio of the active ingredient of the entire pharmaceutical formulation and is expressed as weight/weight (wt/wt).

(1S,2S,3S,5R)-3-((6-(difluoromethyl)-5-fluoro-1,2,3,4-tetrahydroisoquinolin-8-yl)oxy)-5-(4-methyl-7H-pyrrolo [2,3-d]pyrimidin-7-yl)cyclopentane-1,2-diol refers to the compound:

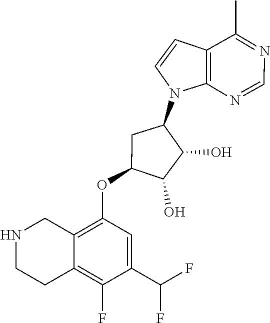 [SMILES: Cc1ncnc2c1ccn2[C@@H]1C[C@H](Oc2cc(C(F)F)c(F)c3c2CNCC3)[C@@H](O)[C@H]1O]

Pharmaceutically acceptable salts of (1S,2S,3S,5R)-3-((6-(difluoromethyl)-5-fluoro-1,2,3,4-tetrahydroisoquinolin-8-yl) oxy)-5-(4-methyl-7H-pyrrolo[2,3-d]pyrimidin-7-yl)cyclopentane-1,2-diol include the acetate, aspartate, benzoate, besylate, bicarbonate/carbonate, bisulphate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate, aluminum, arginine, benzathine, calcium, choline, diethylamine, diolamine, glycine, lysine, magnesium, meglumine, olamine, potassium, sodium, tromethamine, tosylate and zinc salts. For a review on suitable salts, see “Handbook of Pharmaceutical Salts: Properties, Selection, and Use” by Stahl and Wermuth (Wiley-VCH, Weinheim, Germany, 2002), the disclosure of which is incorporated herein by reference in its entirety.

The synthesis of (1S,2S,3S,5R)-3-((6-(difluoromethyl)-5-fluoro-1,2,3,4-tetrahydroisoquinolin-8-yl)oxy)-5-(4-methyl-7H-pyrrolo [2,3-d]pyrimidin-7-yl)cyclopentane-1,2-diol is described in commonly assigned U.S. Pat. No. 10,220,037, the contents of which are incorporated herein by reference in its entirety. In addition, crystalline forms of 1S,2S,3S,5R)-3-((6-(difluoromethyl)-5-fluoro-1,2,3,4-tetrahydroisoquinolin-8-yl) oxy)-5-(4-methyl-7H-pyrrolo[2,3-d]pyrimidin-7-yl)cyclopentane-1,2-diol are described in commonly assigned PCT Publication No. WO2020152557, the contents of which are incorporated herein by reference in its entirety.

### EXAMPLES

Without further elaboration, it is believed that one skilled in the art can, using the preceding description, practice the present invention to its fullest extent. The following detailed examples describe how to prepare the various compounds and/or perform the various processes of the invention and are to be construed as merely illustrative, and not limitations of the preceding disclosure in any way whatsoever. Those skilled in the art will promptly recognize appropriate variations from the procedures both as to reactants and as to reaction conditions and techniques.

### Example 1: Reduced Psoriasis Symptoms in 61 Year Old White Male

A 61 year old white male patient had a history of severe psoriasis for 4-5 years. He had chronic psoriatic lesions on the front and back sides of his torso. The lesions covered 75% of his body, and the patient had difficulty controlling these symptoms despite having used several different medications in the past. The psoriasis symptoms worsened after chemotherapy.

The patient was treated with 1 mg of (1S,2S,3S,5R)-3-((6-(difluoromethyl)-5-fluoro-1,2,3,4-tetrahydroisoquinolin-8-yl) oxy)-5-(4-methyl-7H-pyrrolo[2,3-d]pyrimidin-7-yl)cyclopentane-1,2-diol orally twice a day, for four months.

During the treatment period the patient was questioned about other medical conditions, if any, he was then experiencing. In response the patient indicated that he experienced an improvement in his psoriatic skin lesions and a lessening of related itching within one week of initiating treatment. The patient moreover experienced significant clearing of his psoriatic skin lesions following six weeks treatment. The patient discontinued treatment after four months, for reasons unrelated to his psoriasis. The patient's psoriatic skin lesions remained in remission one month after cessation of treatment. During the time he was treated he did not use any other psoriasis treatments. Photos taken after two and six weeks of treatment document the clearing of psoriasis symptoms. FIGS. 1 and 2 depict said clearing of psoriasis symptoms.

All publications and patent applications cited in the specification are herein incorporated by reference in their entirety. Although the foregoing invention has been described in some detail by way of illustration and example, it will be readily apparent to those of ordinary skill in the art in light of the teachings of this invention that certain changes and modifications may be made thereto without departing from the spirit or scope of the appended claims.

1. A method of treating a subject suffering from an autoimmune disorder comprising administering to said subject in need of a therapeutically effective amount of a PRMT5 inhibitor.

2. The method of claim 1, wherein the PRMT5 inhibitor is (1S,2S,3S,5R)-3-((6-(difluoromethyl)-5-fluoro-1,2,3,4-tetrahydroisoquinolin-8-yl) oxy)-5-(4-methyl-7H-pyrrolo [2,3-d]pyrimidin-7-yl)cyclopentane-1,2-diol or a pharmaceutically acceptable salt thereof.

3. The method of claim 1 [or 2], wherein the autoimmune disorder is selected from the group consisting of psoriasis, atopic dermatitis, alopecia areata, ankylosing spondylitis, asthma, type 1 diabetes, multiple sclerosis, celiac disease, scleroderma, hidradenitis suppurativa, vitiligo, dermatomyositis, systemic lupus erythematosus, rheumatoid arthritis, psoriatic arthritis, and inflammatory bowels disease.

4. The method of claim 3, wherein the autoimmune disorder is psoriasis.

5. The method according to any of the preceding claims, wherein said therapeutically effective amount is administered enterally, parenterally, or topically.

6. (canceled)

7. (canceled)

8. The method according to claim 5, wherein said parenteral administration is administered intravenously or intraarticularly.

9. The method according to claim 5, wherein said topical administration is administered as a solution, a cream, an ointment, a gel, a lotion, a suspension, or an emulsion.

10. The method according claim 2, wherein said subject is administered a therapeutically effective amount from about 0.5 mg to about 120 mg.

11. The method according to claim 10, wherein said therapeutically effective amount is selected from the group consisting of about 0.5 mg, 1 mg, 2 mg, 4 mg, 6 mg, 8 mg, 10 mg, 16 mg, 32 mg, 60 mg, 80 mg, and 120 mg.

12. The method according to claim 11, wherein said therapeutically effective amount is about 12 mg.

13. The method according to claim 11, wherein said therapeutically effective amount is about 10 mg.

14. The method according to claim 11, wherein said therapeutically effective amount is about 8 mg.

15. The method according to claim 11, wherein said therapeutically effective amount is about 6 mg.

16. The method according to claim 11, wherein said therapeutically effective amount is about 4 mg.

17. The method according to claim 11, wherein said therapeutically effective amount is about 1 mg.

18. The method according to claim 2, wherein said therapeutically effective amount is administered once daily (QD).

19. The method according to claim 2, wherein said therapeutically effective amount is administered twice daily (BID).

20. The method according to claim 2, wherein said (1S,2S,3S,5R)-3-((6-(difluoromethyl)-5-fluoro-1,2,3,4-tetrahydroisoquinolin-8-yl)oxy)-5-(4-methyl-7H-pyrrolo [2,3-d]pyrimidin-7-yl)cyclopentane-1,2-diol or a pharmaceutically acceptable salt thereof is administered for 1 to 28 days.

21. The method according to claim 2, wherein said (1S,2S,3S,5R)-3-((6-(difluoromethyl)-5-fluoro-1,2,3,4-tetrahydroisoquinolin-8-yl)oxy)-5-(4-methyl-7H-pyrrolo [2,3-d]pyrimidin-7-yl)cyclopentane-1,2-diol or a pharmaceutically acceptable salt thereof is administered for 1-6 weeks.

22. The method according to claim 2, wherein said (1S,2S,3S,5R)-3-((6-(difluoromethyl)-5-fluoro-1,2,3,4-tetrahydroisoquinolin-8-yl)oxy)-5-(4-methyl-7H-pyrrolo [2,3-d]pyrimidin-7-yl)cyclopentane-1,2-diol or a pharmaceutically acceptable salt thereof is administered for 1-6 months.

23. The method according to claim 22, wherein said (1S,2S,3S,5R)-3-((6-(difluoromethyl)-5-fluoro-1,2,3,4-tetrahydroisoquinolin-8-yl) oxy)-5-(4-methyl-7H-pyrrolo[2,3-d]pyrimidin-7-yl) cyclopentane-1,2-diol or a pharmaceutically acceptable salt thereof is administered for 1-4 months.
